(19) United States
(12) Patent Application Publication
(10) Pub. No.: US 2007/0185413 A1
(43) Pub. Date: Aug. 9, 2007

(54) Guide wire assembly

(75) Inventors: Toshiya Asai, Fujinomiya-shi (JP); Ryuichi Urakawa, Hiratsuka-shi (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, TOKYO (JP)

(21) Appl. No.: 10/592,130

(22) PCT Filed: Dec. 17, 2004
(86) PCT No.: PCT/JP04/18880

§ 371 Date: Sep. 8, 2006

(30) Foreign Application Priority Data
Mar. 15, 2004 (JP) 2004-073593

(51) Int. Cl. A61M 25/00

(52) U.S. Cl. 600/585

(57) ABSTRACT

A guide wire assembly (1) includes a guide wire (2) having a main body section (22), a front-end angular section (21) and a base-end angular section (23). The guide wire assembly (1) further includes a storage section for storing the guide wire (2) inside a hollow section of a substantially annularly wound pipe body (51). The base-end angular section (23) is arranged so that it is dynamically stabilized in elasticity, in a state in which a bent direction thereof is aligned with a direction of curvature of the pipe body (51). When the base-end angular section (23) passes through the pipe body as the guide wire (2) is delivered from the storage section (5), a state is maintained in which the bent direction of the base-end angular section (23) is aligned with the direction of curvature of the pipe body (51), and the direction of the front-end angular section (21) is maintained at a predetermined direction with respect to the storage section (5).

### TECHNICAL FIELD

The present invention relates to a guide wire assembly.

### BACKGROUND ART

As one technique for inserting and indwelling a catheter, such as a central venous catheter, there is the Seldinger technique. The Seldinger technique is a method of inserting a catheter along a guide wire, which is set to indwell within a blood vessel. Therefore, it is very important to insert the guide wire to a target site, and to make the guide wire indwell therein, for making the catheter indwell in the target site.

Insertion of a guide wire into a blood vessel is generally carried out by the following method (following steps [1] to [3])).

[1] insert a cannula into the blood vessel

[2] insert the guide wire into the cannula

[3] push the guide wire forward so as to insert it into the blood vessel

When inserting a guide wire into a blood vessel, use is made of a guide wire assembly (guide wire inserter) having a guide wire stored inside a hollow section of an annularly wound pipe body (see, for example, Japanese Utility Model Registration No. 2601155).

On the other hand, where a front-end section of the guide wire is straight, it may be difficult to insert the guide wire to the target site, due to straying of the guide wire depending on a branched form of the blood vessel. In order to solve this problem, in ordinary cases, including the case described in Patent Document 1 (Japanese Utility Model Registration No. 2601155), a front-end section of the guide wire is formed with a curved angular shape, whereby an improvement in blood vessel selecting performance can be imparted to the front-end section of the guide wire.

However, in the case of a conventional guide wire, such as the guide wire described in Japanese Utility Model Registration No. 2601155, if the guide wire is rotated about its axis while inserting the guide wire to a target site, it becomes impossible to perceive the direction of the front-end angular section of the guide wire. Therefore, the operator may perform an inserting operation while misunderstanding the direction of the front-end angular section; and in such a case, straying of the guide wire, even more than that of a guide wire having a straight front-end section, may be caused, contrary to the expected effect of the front-end angular section.

Further, even if the guide wire is provided with a direction-indicating mark, it is difficult to discriminate the direction using only the naked eye, since the guide wire tends to be extremely thin.

### DISCLOSURE OF THE INVENTION

An object of the present invention is to provide a guide wire assembly which ensures, when inserting a guide wire into a living body, that the operator can perceive easily and assuredly the direction of a front-end angular section of the guide wire, and thus can insert the guide wire while easily and assuredly selecting a target branch blood vessel at a branching portion of the blood vessel.

To attain the above object, according to the present invention, a guide wire assembly is provided, having a guide wire including a main body section, a front-end angular section formed on the front-end side of the main body section and having a portion bent in its natural state, and a base-end angular section formed on the base-end side of the main body section and bent or curved relative to the main body section in its natural state. Further, a storage section is provided, having a pipe body disposed in a curved state substantially over an entire length thereof, the storage section storing the guide wire inside a hollow section of the pipe body. The base-end angular section is configured so as to be dynamically stabilized by the elasticity thereof, in a state in which the bent direction thereof is aligned with the curved direction of the pipe body. During the time that the base-end angular section passes through the pipe body, when the guide wire is delivered from an outlet of the storage section, the bent direction of the base-end angular section is maintained in alignment with the curved direction of the pipe body, whereby the direction of the front-end angular section is maintained at a predetermined direction with respect to the storage section.

This ensures that the direction of the front-end angular section of the guide wire with respect to the storage section is maintained in a predetermined direction during delivery of the guide wire from the storage section. Therefore, when the guide wire is inserted at a target site in a living body, the operator can accurately perceive the direction of the front-end angular section from the direction of the storage section. Accordingly, utilizing the curvature of the front-end angular section, the operator can insert the guide wire while selecting easily and assuredly a target branch blood vessel at a branching portion of the blood vessel.

In addition, in order to attain the above object, according to the present invention, a guide wire assembly is provided, having a guide wire including a main body section, a front-end angular section formed on the front-end side of the main body section and having a portion bent in its natural state, and a base-end angular section formed on the base-end side of the main body section, which is bent or curved relative to the main body section in its natural state. Further, a storage section is provided, including a pipe body having a hollow section with a non-circular cross sectional shape, wherein the storage section stores the guide wire within a lumen of the pipe body.

This ensures that the direction of the front-end angular section of the guide wire is maintained at a predetermined direction with respect to the storage section when the guide wire is delivered from the storage section. Therefore, during insertion of the guide wire to a target site in a living body, the operator can accurately perceive the direction of the front-end angular section through the direction of the storage section. Accordingly, utilizing the curvature of the front-end angular section, the operator can insert the guide wire, while selecting easily and assuredly a target branch blood vessel at a branching portion of the blood vessel.

Further, in the guide wire assembly according to the present invention, during the time that the base-end angular section passes through the pipe body when the guide wire is delivered from an outlet of the storage section, a state is maintained dynamically and stably by the elasticity of the base end angular section, in which the direction of the base-end angular section is aligned with a direction for maximizing the width of the cross section of the hollow section, as viewed in the longitudinal direction of the pipe body. Accordingly, the direction of the front-end angular section is maintained at a predetermined direction with respect to the storage section.

In addition, in the guide wire assembly according to the present invention, the hollow section of the pipe body preferably has an elliptic cross-sectional shape.

Still further, in the guide wire assembly according to the present invention, the pipe body preferably has a groove formed on an inside surface along the longitudinal direction thereof, wherein the width of the hollow section is maximized in the direction of the groove.

In addition, in the guide wire assembly according to the present invention, the cross sectional shape of the hollow section of the pipe body preferably is of a shape such that a portion of a circumferential direction of a circle is enlarged toward the outside in the radial direction.

Still further, in the guide wire assembly according to the present invention, the cross sectional shape of the hollow section of the pipe body preferably is of a shape such that a portion thereof near a central portion of one or both of shorter sides of a rectangle is enlarged toward the outside.

In addition, in the guide wire assembly according to the present invention, the pipe body preferably is disposed in a substantially annularly wound state.

Further, in the guide wire assembly according to the present invention, the distance from the main body section, or an extension line thereof, to a farthest part of the base-end angular section in a natural state is preferably greater than the maximum inside diameter of the pipe body.

### BRIEF DESCRIPTION OF THE DRAWINGS

FIG. 1 is a side view showing a guide wire inserter having a guide wire assembly according to a first embodiment of the present invention.

FIG. 2 is a longitudinal sectional view showing a hub of a puncturing means illustrated in FIG. 1.

FIG. 3 is a side view showing a delivering and introducing member of the guide wire assembly illustrated in FIG. 1.

FIG. 4 is a longitudinal sectional view showing the delivering and introducing member of the guide wire assembly illustrated in FIG. 1.

FIG. 5 is a side view showing, in an enlarged form, the guide wire assembly illustrated in FIG. 1.

FIG. 6 is a schematic longitudinal sectional view of a pipe body and a base-end angular section.

FIG. 7 is a schematic cross sectional view of the pipe body and the base-end angular section.

FIG. 8 is a side view showing another configuration example of the base-end angular section.

FIG. 9 is a cross sectional view of a pipe body of a storage section, in accordance with a second embodiment of the guide wire assembly of the present invention.

FIG. 10 is a cross sectional view of a pipe body of a storage section, in accordance with a third embodiment of the guide wire assembly of the present invention.

FIG. 11 is a cross sectional view of a pipe body of a storage section, in accordance with a fourth embodiment of the guide wire assembly of the present invention.

### BEST MODE FOR CARRYING OUT THE INVENTION

The guide wire assembly according to the present invention shall be described in detail below, based on preferred embodiments thereof as illustrated in the accompanying drawings.

### FIRST EMBODIMENT

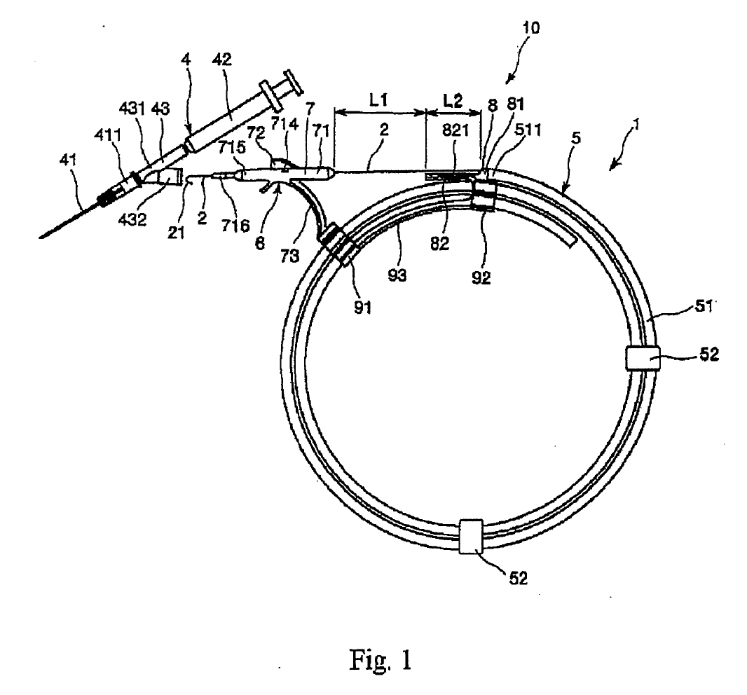

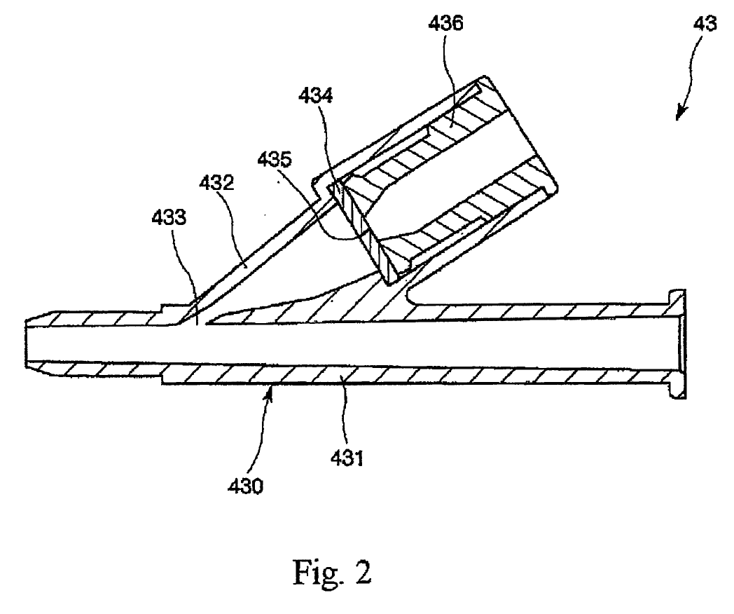

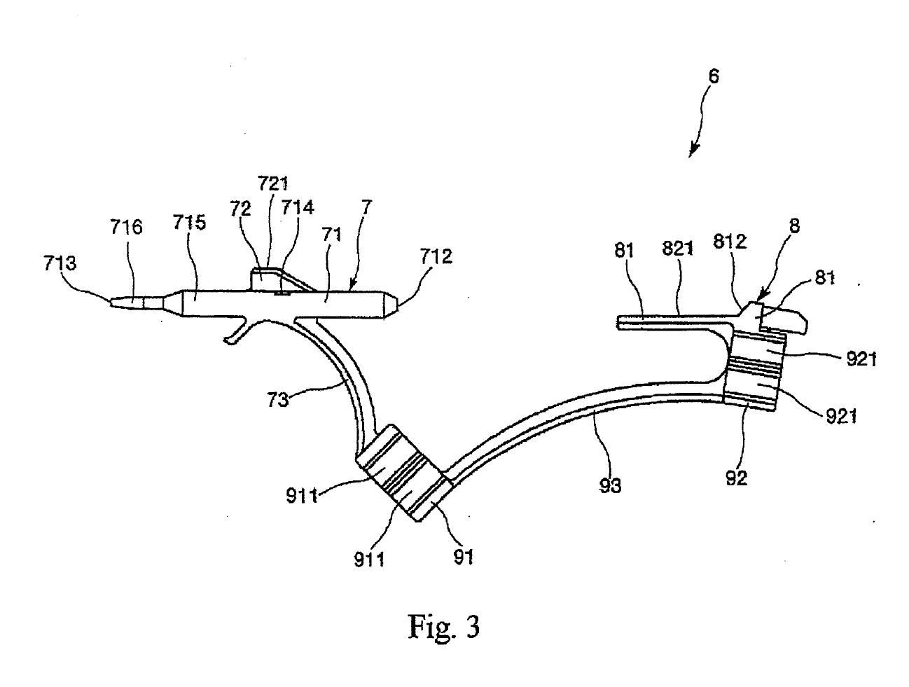

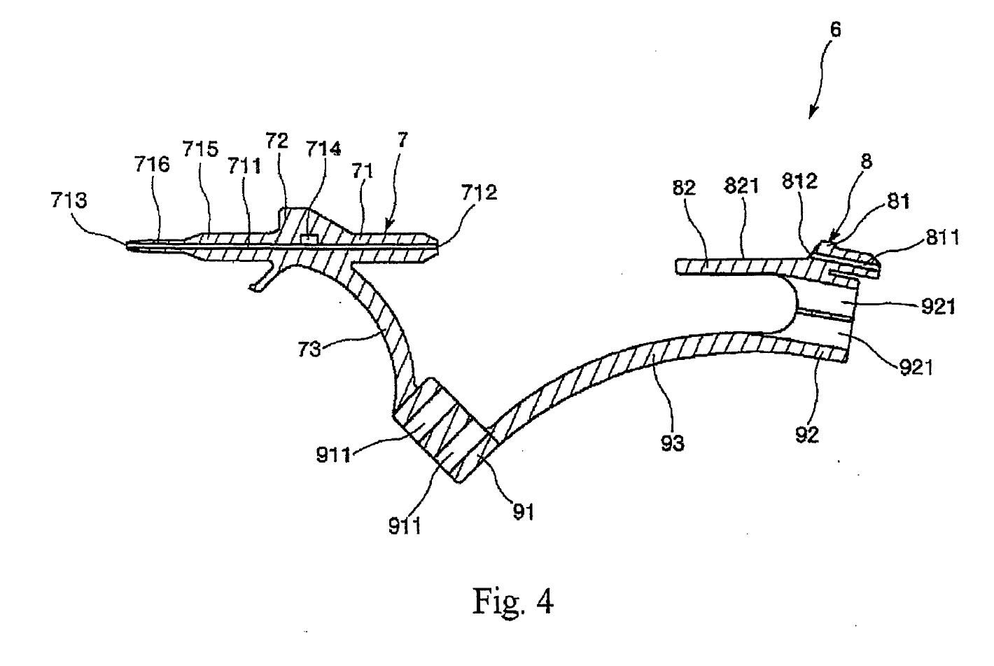

FIG. 1 is a side view showing a guide wire inserter, having a guide wire assembly according to a first embodiment of the present invention; FIG. 2 is a longitudinal sectional view showing a hub of a puncturing means illustrated in FIG. 1; FIG. 3 is a side view showing a delivering and introducing member of the guide wire assembly illustrated in FIG. 1; and FIG. 4 is a longitudinal sectional view showing the delivering and introducing member of the guide wire assembly illustrated in FIG. 1. Incidentally, for convenience in the following descriptions, the left side in FIGS. 1 to 4 shall be referred to as a “front end”, the right side as a “base end”, the upper side as an “upper” portion, and the lower side as a “lower” portion.

The guide wire inserter 10 shown in FIG. 1 is an implement (device) used for percutaneously inserting a guide wire 2 into a blood vessel in a living body, and is composed of the guide wire assembly 1 according to the present invention, together with a puncturing means 4 to which the guide wire assembly 1 is detachably connected.

The guide wire assembly 1 includes the guide wire 2, a storage section (guide wire case) 5 for storing the guide wire 2, and a delivering and introducing member 6, which is detachably attached (fixed) to the storage section 5.

As shown in FIG. 1, the puncturing means 4 includes a hollow puncture needle (hollow needle) 41 having a sharp needle tip, a syringe 42, and a Y-shaped hub (branched connector) 43 provided between the puncture needle 41 and the syringe 42.

As shown in FIG. 2, the hub 43 has a hub main body 430, which is composed of a first tubular body 431, and a second tubular body 432 provided at an intermediate portion of the first tubular body 431. The second tubular body 432 is inclined toward the base-end side, relative to the first tubular body 431, so that the base-end side of the second tubular body 432 is located on the upper portion.

In addition, a side hole (through-hole) 433 is provided at an intermediate portion of the first tubular body 431, whereby an inside portion (lumen) of the first tubular body 431 communicates with an inside portion (lumen) of the second tubular body 432.

The size of the side hole 433 is not particularly limited, and may be appropriately set according to various conditions, such as the outside diameter of the guide wire 2 to be used. When the side hole 433 is substantially circular in cross sectional shape, the diameter of the side hole 433 should preferably be about 0.7 to 3.0 mm, or more preferably, about 0.8 to 2.0 mm.

This ensures that positioning of the guide wire 2 can be properly regulated, and that the guide wire 2 can be moved (inserted) more smoothly in the direction of the puncture needle 41.

A hub 411, which is provided at a base-end portion of the puncture needle 41, is detachably connected to a front-end portion of the first tubular body 431, in order to ensure that the inside portion (lumen) of the puncture needle 41, the inside portion (lumen) of the first tubular body 431, and the inside portion (lumen) of the second tubular body 432 all communicate with one another.

In addition, a front-end portion of a syringe 42 is detachably connected to a base-end portion of the first tubular body 431.

A valve body 434 provided with a straight line-shaped slit (straight line slit) 435 is disposed inside of the second tubular body 432 of the hub main body 430 at an intermediate portion thereof. A reduced diameter portion (small diameter part) 716 of a front-end section 715 of an introducing section 7 of the guide wire assembly 1 (to be described later) can be inserted from the base-end side to the front-end side of the second tubular body 432 through the slit 435 in the valve body 434. The valve body 434 maintains liquid-tightness (and gas-tightness) between the front-end side (first tubular body 431 side) and the base-end side (exterior) of the second tubular body 432.

Incidentally, the shape of the slit 435 provided in the valve body 434 is not limited to a straight line shape, but other shapes, for example a cross shape (cross slit), may also be adopted.

In addition, the valve body 434 may, for example, have a first cut opening on one end face thereof but not on the other end face, wherein a second cut intersects with the first cut at an inside portion thereof, and wherein the second cut opens on the other end face but not on the one end face (i.e., a valve body provided with a substantially cross-shaped slit composed of straight line slits formed to alternately intersect with each other).

Further, examples of materials constituting the valve body 434 include various rubbers such as silicone rubber, natural rubber, etc., and elastic materials (soft materials are particularly preferable) such as thermoplastic elastomers, etc.

In addition, a tubular cap (female connector) 436 is disposed on an inside portion of the second tubular body 432 on the base-end side of the valve body 434. The inside shape of the cap 436 corresponds to the outside shape of the front-end section 715 of the introducing section 7.

The introducing section 7 of the guide wire assembly 1 is inserted, starting from the front-end side thereof, into the base-end portion of the second tubular body 432, namely, into the cap 436. This results in the introducing section 7 being detachably connected to the hub 43. Therefore, the second tubular body 432, the cap 436 and the valve body 434 together constitute a connection section (connection port).

As shown in FIG. 1, the guide wire assembly 1 is formed by the guide wire 2, the storage section 5 for storing the guide wire 2, and the delivering and introducing member 6, which is detachably attached (fixed) to the storage section 5.

The structure, materials and the like of the guide wire 2 are not particularly limited, however, it is preferable, for example, for the guide wire 2 to include a core member composed of a superelastic material (superelastic wire), wherein a coil is provided at a front-end portion of the core member.

When the coil is provided at the front-end portion of the core member composed of a superelastic material, sufficient flexibility can be obtained, and the diameter thereof can be maintained at a predetermined value.

The material constituting the core member is not particularly limited, however, superelastic alloys, for example Ni—Ti based alloys, and the like are preferable.

On the other hand, the material constituting the coil is not particularly limited, however, metallic materials, for example stainless steel, and the like are preferable.

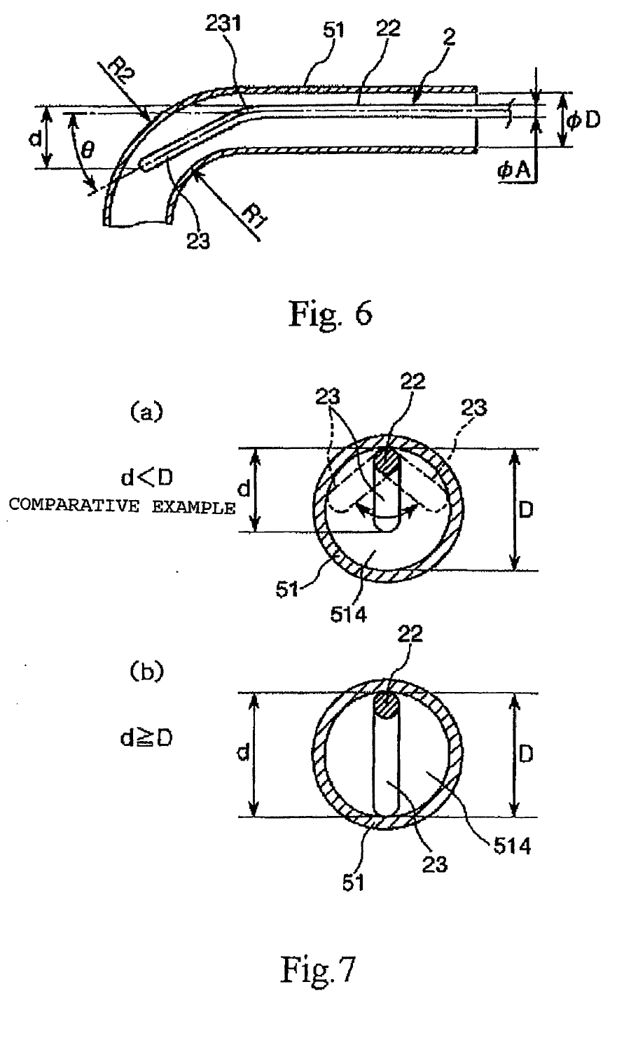

In addition, the outside diameter A (see FIG. 6) of the guide wire 2 is not particularly limited, but is preferably not more than 1.0 mm, or more preferably, lies in a range of about 0.3 to 0.9 mm.

A front-end angular section 21, curved in a J shape in its natural state (in a condition where no external force is exerted thereon), is formed in the vicinity of the front end of the guide wire 2. During the course of inserting the guide wire 2 to a target site within a living body, by aligning the direction of the front-end angular section 21 with the direction of a target branch blood vessel, it is possible to assuredly select the target branch blood vessel at a branching portion of the blood vessel. Incidentally, the shape of the front-end angular section 21 is not limited to the shape shown in the figures, but may be of other types as well.

The storage section 5 is composed of a flexible pipe body (tube) 51, so that the guide wire 2 can be stored inside a hollow section (lumen) 514 of the pipe body 51. The pipe body 51 is bundled while being wound in a substantially annular (circular) form. Specifically, the pipe body 51 is bundled while being wound substantially along the circumference of a circle.

The pipe body 51 is held in an annularly wound and bundled state by two clips (holding members) 52, each of which are provided with two through-holes therein.

Incidentally, the number of loops (i.e., the looped amount) of the pipe body 51 is not particularly limited, although in the illustrated example, the number of loops is approximately two.

In addition, the hollow section 514 of the pipe body 51 has a substantially circular cross sectional shape.

As shown in FIGS. 3 and 4, the delivering and introducing member 6 includes a delivering section (guide wire delivering section) 8 for delivering therethrough the guide wire 2 stored in the storage section 5, and an introducing section (guide wire introducing section) 7 for receiving the guide wire 2 delivered from the delivering section 8 and for insertion of the guide wire 2 therethrough. The delivering section 8 and the introducing section 7 are spaced apart by a predetermined distance.

The delivering section 8 has a tubular delivering section main body 81 and a plate-like pedestal (operating section) 82. The delivering section 8 (particularly, an outlet 812 thereof) is located substantially on the circular circumference of the storage section 5. The front-end side opening (front-end opening) of a hole (through-hole) 811, which is formed in the delivering section main body 81, constitutes an outlet 812 for the guide wire 2. The guide wire 2 is passed through the hole 811 in the delivering section main body 81, so as to be delivered through the outlet 812 toward the introducing section 7.

The pedestal 82 projects from a position in the vicinity of the outlet 812 of the delivering section main body 81, and on a lower side of the outlet 812, toward a position in the vicinity of an inlet 712 of the introducing section 7 (to be described later) and on a lower side of the inlet 712. In other words, the pedestal 82 is located in the vicinity of the guide wire 2 and the outlet 812, while being disposed between the guide wire 2 and the storage section 5.

An upper-side surface (upper face) of the pedestal 82 faces to the guide wire 2 (such that the guide wire 2 is located between the delivering section main body 81 and the introducing section 7) and constitutes a contact surface (opposite surface) 821, with which the guide wire 2 and a finger of the user can make contact.

The pedestal 82 enables an operation whereby the guide wire 2 can be pulled toward the base end side, and other similar operations that can be easily carried out using only one hand.

In addition, as shown in FIG. 1, a front-end section 511 of the pipe body 51 is connected to (i.e., inserted into) the base-end side of the delivering section main body 81, whereby the hole 811 and the lumen of the pipe body 51 in the storage section 5 communicate with each other.

The space between the delivering section 8 and the introducing section 7, specifically the space between the front end of the pedestal 82 of the delivering section 8 and the base end of the introducing section 7, as well as the surrounding area therebetween, constitutes a space (gripping space) for operating the guide wire 2 by gripping it with one's fingers.

Therefore, a spacing L1 between the delivering section 8 and the introducing section 7, specifically between the front end of the pedestal 82 of the delivering section 8 and the base end of the introducing section 7, is set so that the fingers of the user (operator) can enter between the front end of the pedestal 82 and the base end of the introducing section 7.

More specifically, the spacing L1 is preferably about 20 to 80 mm, and more preferably, about 30 to 70 mm.

In addition, the length L2 of the pedestal 82 in the longitudinal direction (the left to right direction as shown in FIG. 1) is not particularly limited, but is preferably about 5 to 50 mm, and more preferably, about 10 to 30 mm.

As shown in FIGS. 3 and 4, the introducing section 7 includes a tubular introducing section main body 71. A base-end side opening (base-end opening) of the hole (through-hole) 711 formed in the introducing section main body 71 constitutes the inlet 712 for the guide wire 2, and a front-end side opening (front-end opening) constitutes an outlet 713 for the guide wire 2. The guide wire 2, which is delivered from the outlet 812 of the guide wire delivering section 8, passes through the hole 711 formed in the introducing section main body 71. More specifically, the guide wire 2, which is delivered from the guide wire delivering section 8, is inserted from the inlet 712 and is delivered from the outlet 713.

In addition, a front-end section 715 of the introducing section main body 71 is provided on a frontal end side thereof, with a reduced diameter portion (small diameter part) 716 having a reduced outside diameter.

Further, a central portion of the introducing section main body 71 is provided with a plate-like wall part (barrier) 72 disposed on an upper portion thereof. A side view of the wall part 72 is substantially trapezoidal (see FIGS. 3 and 4). The wall part 72 is provided with a rib 721 projecting from a back side toward a face side of the sheet, as shown in FIG. 3, extending over a range from an upper end to a base end thereof (in FIGS. 3 and 4, on the upper and right-hand sides of the trapezoid). The rib 721 projects to a position extending beyond a hole 714, which shall be described later.

In addition, the central portion of the introducing section main body 71 is provided with a hole 714 on an upper portion thereof. The hole 714 communicates with the hole 711 on one side thereof, while the other side of the hole 714 is open to the exterior, at a position corresponding to the wall part 72 of the introducing section main body 71, proximate to the rib 721 side thereof (i.e., the face side of the sheet as shown in FIG. 3) relative to the wall part 72.

Due to the hole 714, blood can be released through the hole 714 in the event that blood flows reversely via the puncturing means 4, and accordingly, blood can be prevented (inhibited) from leaking into an operating region (i.e., into a vicinity of the operator's hand). In addition, in case the puncture needle 41 of the puncturing means 4 punctures an artery by mistake, contamination of the operating region can be prevented or restrained, and further, mistaken puncturing of the artery can be inferred (detected) from the magnitude of blood flow that flows out of the hole 714.

In addition, when the wall part 72 is provided as described above, the wall part 72 serves as a barrier, in the event that blood jets out of the hole 714, whereby blood can be prevented (inhibited) from scattering (particularly, scattering into the operating region).

Further, the central portion of the introducing section main body 71 is provided with an arm part 73 at a lower portion thereof, wherein the arm part 73 is provided with a fixing part (first fixing part) 91 disposed at a lower end portion thereof.

The fixing part 91 is provided with two grooves 911, whereby the pipe body 51 in the storage section 5 is detachably fitted into each of the grooves 911, as shown in FIG. 1. As a result, the introducing section 7 is detachably fixed to the storage section 5 through the fixing part 91, and the pipe body 51 is held by the fixing part 91 while being annularly wound and bundled.

In addition, a fixing part (second fixing part) 92 is provided at a lower portion of the delivering section main body 81 of the delivering section 8.

The fixing part 92 is provided with two grooves 921 therein, whereby the pipe body 51 in the storage section 5 is detachably fitted into each of the grooves 921, as shown in FIG. 1. As a result, the delivering section 8 is detachably fixed to the storage section 5 through the fixing part 92, at a position different from the position where the introducing section 7 is detachably fixed, and the pipe body 51 is held by the fixing part 92 while being annularly wound and bundled.

In addition, the fixing part 91 and the fixing part 92 are connected to each other by a connecting part 93. As shown in FIG. 1, the connecting part 93 has a circular arc shape that is substantially the same as the shape of the storage section 5 (pipe body 51). Namely, the fixing part 91 and the fixing part 92 are connected to each other while lying alongside the circular arc of the storage section 5 (pipe body 51) through the connecting part 93.

Such a structure ensures that the spacing L1 between the front end of the pedestal 82 of the delivering section 8 and the base end of the introducing section 7 is constantly maintained at a predetermined distance. Further, since the connecting part 93 has an arcuate shape, the connecting part 93 does not constitute an obstacle.

While, in this embodiment, the delivering and introducing member 6 is formed integrally (i.e., formed as a single member), the configuration is not restricted in this manner; for example, the delivering and introducing member 6 may also be formed by joining together a plurality of separate members.

In addition, the material constituting the delivering and introducing member 6 is not particularly limited. Examples of materials that can be used include polyolefins, such as polyethylene, polypropylene, ethylene-propylene copolymer, etc., polystyrene, polycarbonate, acrylic resins, acrylonitrile-butadiene-styrene copolymers (ABS resins), acrylonitrile-styrene copolymers (AS resins), and copolymers, blends, polymer alloys and the like, based on such polymers.

Next, with reference to FIG. 1, an example of the method for using the guide wire inserter 10 (guide wire assembly 1) shall be described below. In this case, a representative example of an operating procedure for inserting a central venous catheter (IVH catheter) shall be described.

First, the puncture needle 41 of the puncturing means 4 is used to puncture a predetermined portion of a patient, wherein the needle tip of the puncture needle 41 is inserted into a blood vessel (vein).

Then, blood is aspirated by the syringe 42, in order to confirm that the needle tip of the puncture needle 41 is located within the blood vessel.

Subsequently, using one's finger, the guide wire 2 is pressed against the contact surface 821 of the pedestal 82 of the delivering section 8, and the guide wire 2 is pulled toward the base-end side, thereby locating a portion or the entirety of the front-end angular section 21 of the guide wire 2 in the introducing section 7. As a result, the J-shaped front-end angular section 21 of the guide wire 2 does not form an obstacle.

Next, the introducing section 7 of the guide wire assembly 1 is connected to the second tubular body 432 of the hub 43 of the puncturing means 4.

As a result, the reduced diameter portion 716 of the front-end section 715 of the introducing section 7 is inserted through the slit 435 in the valve body 434 and into the second tubular body 432, on the front-end side of the valve body 434. In addition, the valve body 434 maintains liquid-tightness (gas-tightness) between the front-end side (the first tubular body 431 side) and the base-end side (exterior) of the second tubular body 432.

Next, an operation is carried out in order to insert the guide wire 2 into the blood vessel.

Specifically, the guide wire 2, which is located between the delivering section 8 and the introducing section 7, is gripped with the fingers and delivered.

As a result, the guide wire 2 stored in the storage section 5 is delivered from the outlet 812 of the delivering section 8, whereby the front-end angular section 21 of the guide wire 2 passes through the lumen of the second tubular body 432 of the hub 43, as shown in FIG. 2, and further passes through the side hole 433, the lumen of the first tubular body 431, and the lumen of the puncture needle 41 of the puncturing means 4, whereupon the front-end angular section 21 of the guide wire 2 projects from the needle tip of the puncture needle 41, so as to be inserted into the blood vessel.

Subsequently, while the guide wire 2 is left in the blood vessel, the puncture needle 41 is withdrawn, so that the guide wire 2 becomes detached from the guide wire assembly 1 and is made to indwell within the blood vessel.

Next, a catheter (not shown) is inserted along the guide wire 2 into the blood vessel.

Subsequently, while the catheter is left in the blood vessel, the guide wire 2 is withdrawn, whereby the catheter is made to indwell within the blood vessel.

As has been described above, according to the guide wire assembly 1 of this embodiment, the delivering section 8 and the introducing section 7 are spaced apart, and a space (gripping space) is formed, whereby the guide wire 2 may be gripped and operated on using one's fingers, so that the guide wire 2 can be manipulated easily and assuredly with one hand while gripping it directly with the fingers.

Specifically, since the guide wire 2 can be gripped and manipulated with the fingers, a sense of inserting the guide wire 2 can be securely obtained during insertion of the guide wire 2, so that slippage of the guide wire 2 can be prevented or restrained, and the guide wire 2 can be manipulated with ease.

In addition, since the delivering section 8 and the introducing section 7 are fixed respectively to different portions of the storage section 5, chattering or unsteadiness of the guide wire assembly 1 is prevented, and the guide wire assembly 1 similarly can be handled with ease.

Further, since the delivering section 8 (particularly, the outlet 812 thereof) is located substantially along the circular circumference of the storage section 5, operation thereof is easy and the size of the guide wire assembly 1 can be reduced.

Incidentally, in this embodiment, the fixing part 91 and the fixing part 92 need not necessarily be connected to each other (i.e., such elements may be made separate from each other).

In addition, the guide wire assembly 1 is preferably provided with a fixture (fixing means) for reversibly connecting and fixing the guide wire assembly 1 to the puncturing means 4. Thus, it is possible to carry out insertion operations (gripping operations) of the guide wire 2 with ease.

Further, the guide wire assembly 1 is preferably provided with a fixing means such as, for example, a stopper, for reversibly fixing the guide wire 2. This makes it possible to more assuredly prevent (inhibit) the guide wire 2 from flying out of the storage section 5 before insertion of the guide wire 2.

While one example of a method for using the guide wire assembly 1 according to the present embodiment has been described above, the delivering and introducing member 6 in the guide wire assembly of the present invention is not limited to the configuration illustrated in the figures, and other configurations are also possible. For example, the delivering section 8 and the introducing section 7 need not necessarily be spaced apart from each other. Furthermore, the guide wire assembly 1 of the present invention need not necessarily include a member corresponding to the delivering and introducing member 6 of the present embodiment.

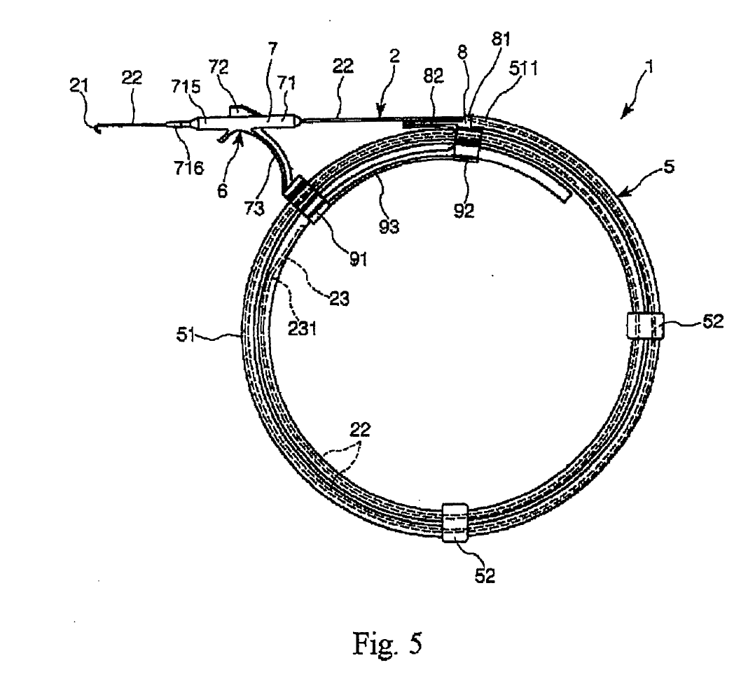

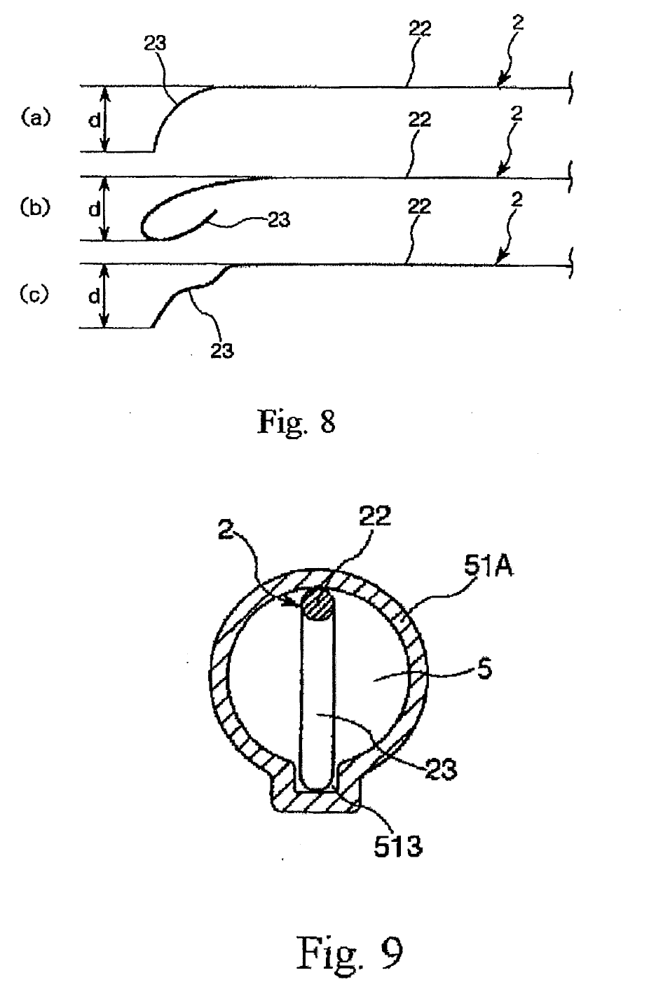

FIG. 5 is a side view showing, in an enlarged form, the wire guide assembly shown in FIG. 1; FIG. 6 is a schematic longitudinal sectional view of a pipe body and a base-end angular section; FIG. 7 is a schematic cross sectional view of the pipe body and the base-end angular section; and FIG. 8 is a side view showing another configuration example of the base-end angular section.

Next, based on the above figures, the guide wire assembly 1 according to the present invention shall be described in greater detail below.

As shown in FIG. 5, the guide wire 2 includes a main body section 22, which is substantially straight in its natural state, wherein the above-mentioned front-end angular section is formed on the front-end side of the main body section 22.

Further, the guide wire 2 includes a base-end angular section 23 formed on the base-end side of the main body section 22. The base-end angular section 23, in its natural state (i.e., in a condition where no external forces are exerted thereon), is bent in one direction relative to the main body section 22 through a bent part 231.

When the guide wire 2 is rotated about its axis, in a condition in which the base-end angular section 23 resides within the storage section 5 (pipe body 51), the base-end angular section 23 is dynamically stabilized in the state shown in FIG. 5, in which the bent direction of the base-end angular section 23 is aligned with the direction of curvature of the pipe body 51. In other words, in this state, elastic energy accumulated in the vicinity of the base-end angular section 23 is minimized and stabilized, and therefore, even if the guide wire 2 is gripped by hand and rotated about its axis, so that the bent direction of the base-end angular section 23 is non-aligned with the direction of curvature the pipe body 51, releasing the hand from the guide wire 2 results in the base-end angular section 23 being automatically returned to the state shown in FIG. 5, due to the elastic force (i.e., a restoring force for returning the guide wire to its original shape) in the vicinity of the base-end angular section 23.

In the guide wire assembly 1 of the present invention, as a result of the above action, during the time that the base-end angular section 23 passes through the storage section 5 (pipe body 51), as the guide wire 2 is delivered from the outlet 812 of the storage section 5, a state is maintained in which the bent direction of the base-end angular section 23 is aligned with the curved direction of the pipe body 51, and the base-end angular section 23 is prevented from rotating about its axis. Since the main body section 22 of the guide wire 2 has a torque transmission performance (torsional rigidity), such that the front-end angular section 21 is rotated attendant on the rotation of the base-end angular section 23, by preventing the base-end angular section 23 from rotating inside the storage section 5 (pipe body 51), it is also assured that the direction of the front-end angular section 21 with respect to the storage section 5 is maintained in the directional state shown in FIG. 5 while the guide wire 2 is delivered from the storage section 5.

As described above, in the guide wire assembly 1 of the present invention, when the guide wire 2 is delivered from the storage section 5, the direction of the front-end angular section 21 is maintained at a predetermined orientation with respect to the direction of the storage section 5. Therefore, during insertion of the guide wire 2 to a target site within a living body, the direction of the front-end angular section 21 can be accurately perceived from the direction of the storage section 5. Accordingly, utilizing the curvature of the front-end angular section 21, the guide wire 2 can be inserted while easily and assuredly selecting a target branch blood vessel at a branching portion of a blood vessel.

In order to more securely achieve the above-mentioned effect, as shown in FIG. 6, the base-end angular section is preferably shaped and sized so that, in its natural state, the distance d from the main body section 22, or an extension line thereof, to the farthest portion of the base-end angular section 23 is not less than the inside diameter (maximum inside diameter) D of the pipe body 51. When d is less than D (d<D) as shown in the comparative example of FIG. 7(a), the base-end angular section 23 can rotate within the pipe body 51. However, when d is greater than or equal to D (d≧D) as shown in FIG. 7(b), the base-end angular section 23 is prevented from rotating within the pipe body 51.

In addition, when the radius of curvature of the pipe wall on the circumferential inner side of the pipe body 51 is R1, and the radius of curvature of the pipe wall on the circumferential outer side of the pipe body 51 is R2 (wherein R1<R2), as shown in FIG. 6, the guide wire 2 preferably has a restoring property, such that when a load is exerted on a straight portion of the guide wire 2, so as to curve the portion at a radius of curvature of less than R1, and then the load is released, the radius of curvature of the portion returns to a value of not less than R2. Incidentally, FIG. 6 is a schematic view, in which the curvature of the pipe body 51 has been exaggerated.

Further, in the case of the base-end angular section 23, which is shaped as shown in FIGS. 5 and 6, in its natural state, the angle of inclination θ (see FIG. 6) of the base-end angular section 23 relative to the main body section 22 is not particularly limited, but is preferably 3° to 45°, and more preferably, 5° to 15°.

Incidentally, the shape of the base-end angular section 23 is not limited to the shape shown in FIGS. 5 and 6. For example, the shape may be any of the curved shapes shown in FIGS. 8(a), 8(b) and 8(c). In each of these figures, the distance d is the distance at the position shown.

### SECOND EMBODIMENT

FIG. 9 is a cross sectional view of a pipe body of a storage section, in accordance with a second embodiment of the guide wire assembly of the present invention. The second embodiment of the guide wire assembly of the present invention shall be described below with reference to the figure, referring mainly to differences from the first embodiment, and while omitting from description items that are the same as those already addressed above.

This embodiment is the same as the first embodiment above, except that the cross sectional shape of the pipe body 51A of the storage section 5 is different.

As shown in FIG. 9, according to this embodiment, the cross sectional shape of the hollow section 514 of the pipe body 51A is non-circular. Specifically, the cross sectional shape of the hollow section 514 of the pipe body 51A is such that a part thereof (i.e., a lower part as shown in FIG. 9) in the circumferential direction of a circle is enlarged toward the outside in the radial direction. The enlarged part constitutes a groove 513, which is formed on an inside surface of the pipe body 51A along a longitudinal direction. The width of the groove 513 is greater than the wire diameter of the guide wire 2, so that the guide wire 2 can be inserted into the groove 513. Incidentally, the same or a similar groove may also be formed on an opposite side of the groove 513 with respect to the center of the pipe body 51A.

This configuration ensures that the width (inside diameter) of the hollow section 514 of the pipe body 51A is maximum in a direction where the groove 513 exists (i.e., in the vertical direction as shown in FIG. 9), whereas, in other directions, the width (inside diameter) of the hollow section 514 of the pipe body 51A is smaller than the maximum value.

Incidentally, as in the first embodiment, in its natural state, the distance d from the main body section 22, or an extension line thereof, to the farthest portion of the base-end angular section 23 is not less than the maximum width (maximum inside diameter) of the hollow section 514 of the pipe body 51A.

When, as described above, the base-end angular section 23 is present in the pipe body 51A, a dynamically stable state (i.e., the state shown in FIG. 9) is provided, in which the direction of the base-end angular section 23 is aligned with a direction (the vertical direction as shown in FIG. 9) in which the width (inside diameter) of the cross section of the hollow section 514 of the pipe body 51A is maximum. This state is maintained while the base-end angular section 23 passes through the storage section 5 (pipe body 51A), whereby the guide wire 2 is prevented from rotating about its axis. Therefore, the direction of the front-end angular section 21 can be maintained at a predetermined orientation with respect to the storage section 5 during periods when the guide wire 2 is delivered from the storage section 5. Accordingly, in this embodiment, the same effects as those of the first embodiment can be obtained.

Incidentally, in this embodiment, the pipe body 51A need not necessarily be arranged in a curved shape, but may also be arranged in a rectilinear form.

### THIRD EMBODIMENT

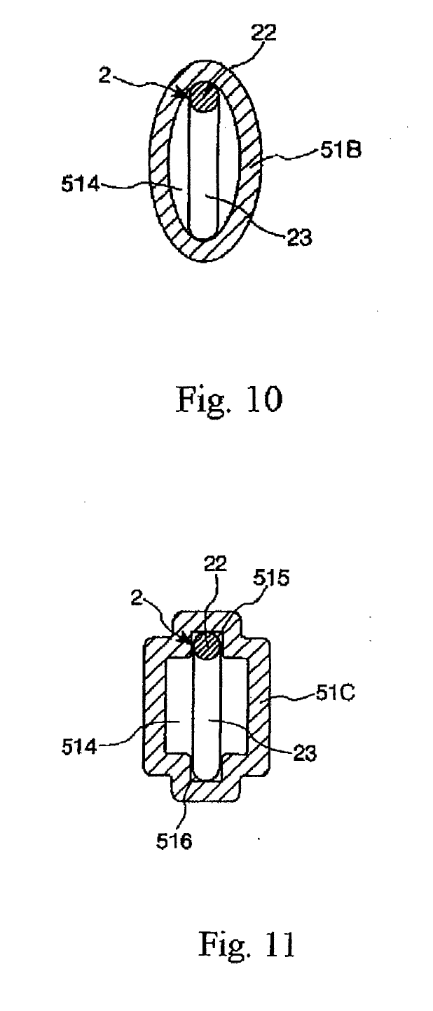

FIG. 10 is a cross sectional view of a pipe body of a storage section, in accordance with a third embodiment of the guide wire assembly of the present invention. The third embodiment of the guide wire assembly of the present invention shall be described below with reference to the figure, referring mainly to differences from the first embodiment, and while omitting from description items that are the same as those already addressed above.

This embodiment is the same as the first embodiment above, except that the cross sectional shape of the pipe body 51B of the storage section 5 is different.

As shown in FIG. 10, the cross sectional shape of the hollow section 514 of the pipe body 51B in this embodiment is non-circular. Specifically, the cross sectional shape of the hollow section 514 of the pipe body 51B is an ellipse having a major axis in the vertical direction in FIG. 10, and a minor axis in the left-right direction in FIG. 10.

This configuration ensures that the width (inside diameter) of the hollow section 514 of the pipe body 51B is maximum in the vertical direction shown in FIG. 10, whereas, in other directions, the width (inside diameter) of the hollow section 514 of the pipe body 51B is smaller than the maximum value.

Incidentally, as in the first embodiment, in its natural state, the distance d from the main body section 22, or an extension line thereof, to the farthest portion of the base-end angular section 23 is not less than the maximum width (maximum inside diameter) of the hollow section 514 of the pipe body 51B.

When, as described above, the base-end angular section 23 is present in the pipe body 51B, a dynamically stable state (i.e., the state shown in FIG. 10) is provided, in which the direction of the base-end angular section 23 is aligned with a direction (the vertical direction as shown in FIG. 10) in which the width (inside diameter) of the cross section of the hollow section 514 of the pipe body 51B is maximum. This state is maintained while the base-end angular section 23 passes through the storage section 5 (pipe body 51B), whereby the guide wire 2 is prevented from rotating about its axis. Therefore, the direction of the front-end angular section 21 can be maintained at a predetermined orientation with respect to the storage section 5 during periods when the guide wire 2 is delivered from the storage section 5. Accordingly, in this embodiment, the same effects as those of the first embodiment above can be obtained.

Incidentally, in this embodiment, the pipe body 51B need not necessarily be arranged in a curved shape, but may also be arranged in a rectilinear form.

### FOURTH EMBODIMENT

FIG. 11 is a cross sectional view of a pipe body of the storage section, in accordance with a fourth embodiment of the guide wire assembly of the present invention. The fourth embodiment of the guide wire assembly of the present invention shall be described below with reference to the figure, while referring mainly to differences from the first embodiment, and while omitting items that are the same as those already addressed above.

This embodiment is the same as the first embodiment above, except that the cross sectional shape of the pipe body 51C of the storage section 5 is different.

As shown in FIG. 11, according to this embodiment, the cross sectional shape of the hollow section 514 of the pipe body 51C is non-circular. Specifically, the cross sectional shape of the hollow section 514 of the pipe body 51C is such that portions thereof, which are near to the central portions of each of the short sides of a rectangle, are enlarged toward the outside. As a result of such enlarged portions, the inside surface of the pipe body 51C is provided with grooves 515 and 516 extending in the longitudinal direction, at upper and lower positions, as shown in FIG. 11. The width of the grooves 515, 516 is greater than the wire diameter of the guide wire 2, so that the guide wire 2 can be inserted into the grooves 515, 516. Incidentally, a structure may also be adopted in which only one of the grooves 515 and 516 is formed.

The above configuration ensures that the width (inside diameter) of the hollow section 514 of the pipe body 51C is maximum in the direction where the grooves 515, 516 are formed (i.e., in the vertical direction shown in FIG. 11), whereas, in other directions, the width (inside diameter) of the hollow section 514 of the pipe body 51C is smaller than the maximum value.

Incidentally, as in the first embodiment, in its natural state, the distance d from the main body section 22 or an extension line thereof to the farthest portion of the base-end angular section 23 is not less than the maximum width (maximum inside diameter) of the hollow section 514 of the pipe body 51C.

When the base-end angular section 23 is present in the pipe body 51C, as described above, a dynamically stable state is provided (i.e., the state shown in FIG. 11), in which the direction of the base-end angular section 23 is aligned with a direction (i.e., the vertical direction in FIG. 11) in which a width (inside diameter) of the cross section of the hollow section 514 of the pipe body 51C is maximum. Such a state is maintained when the base-end angular section 23 passes through the storage section 5 (pipe body 51C), so that the guide wire 2 is prevented from rotating about its axis, and the direction of the front-end angular section 21 is maintained at a predetermined orientation with respect to the storage section 5 during periods when the guide wire 2 is delivered from the storage section 5. Accordingly, the same effects as those of the first embodiment can be obtained.

Incidentally, in this embodiment, the pipe body 51C need not necessarily be arranged with a curved shape, but may be arranged in a rectilinear form.

While the guide wire assembly according to the present invention has been described above with reference to the embodiments shown in the drawings, the present invention is not limited to the illustrated embodiments, and the configurations of the respective parts or elements thereof may be replaced by other arbitrary configurations having the same or equivalent functions.

In addition, the manner of use of the guide wire assembly of the present invention is not particularly limited. For example, the guide wire assembly may be used for inserting a guide wire that serves as a guide for various types of catheters, such as a central venous catheter.

Further, while in the first embodiment the direction of curvature of the pipe body 51 of the storage section 5 has been described as being a substantially annular shape, which is constant over the entire length thereof, the direction of curvature of the pipe body 51 may alternatively have an S shape at intermediate positions in the longitudinal direction. Such a shape ensures that the direction of the front-end angular section 21 can automatically be changed according to the delivery amount of the guide wire 2 from the storage section 5. In application, when a configuration is prepared in which the curved direction of the pipe body 51 is changed along the longitudinal direction according to a branching form of a blood vessel in a living body, the direction of the front-end angular section 21 can automatically select the direction of the target blood vessel according to a depth of insertion of the guide wire 2 within the living body.

In addition, in situations where the cross sectional shape of the hollow section 514 of the pipe body 51 is non-circular, as in the second to fourth embodiments, such a shape is not limited to the shapes described in the second to fourth embodiments. For example, the shape of the hollow section 514 may be any of triangles, tetragons, pentagons, hexagons or other multi-apexed polygons and regular polygons, semi-circles (semi-ellipses), etc.

Further, even when the cross sectional shape of the hollow section 514 of the pipe body 51 is non-circular, as in the second to fourth embodiments, the outer periphery (outside wall surface) of the pipe body 51 can still be made circular in cross section.

### INDUSTRIAL APPLICABILITY

A guide wire assembly is provided, in which the direction of a front-end angular section of the guide wire with respect to the direction of the storage section can be maintained at a predetermined orientation while the guide wire is delivered from the storage section. Therefore, during insertion of the guide wire to a target site within a living body, an operator can accurately perceive the direction of the front-end angular section from the orientation of the storage section. Accordingly, by utilizing the curvature of the front-end angular section, the operator can insert the guide wire while easily and assuredly selecting a target branch blood vessel at a branching portion of a blood vessel. Therefore, the guide wire assembly of the present invention demonstrates industrial applicability.

1. A guide wire assembly comprising: a guide wire including a main body section, a front-end angular section formed on a front-end side of said main body section and having a portion bent in its natural state, and a base-end angular section formed on a base-end side of said main body section, and which is bent or curved relative to said main body section in its natural state; and a storage section having a pipe body that is curved over substantially an entire length thereof, said storage section storing said guide wire inside a hollow section of said pipe body, wherein said base-end angular section is dynamically stabilized by its elasticity, in a state in which a direction of bending of said base-end angular section is aligned with a direction of curvature of said pipe body, and wherein, when said base-end angular section passes through said pipe body as said guide wire is delivered from an outlet of said storage section, said state, in which the direction of bending of said base-end angular section is aligned with the direction of curvature of said pipe body, is maintained, whereby the direction of said front-end angular section is maintained at a predetermined orientation with respect to said storage section.

2. A guide wire assembly comprising: a guide wire including a main body section, a front-end angular section formed on a front-end side of said main body section and having a portion bent in its natural state, and a base-end angular section formed on a base-end side of said main body section, and which is bent or curved relative to said main body section in its natural state; and a storage section including a pipe body having a hollow section with a non-circular cross sectional shape, said storage section storing said guide wire in a lumen of said pipe body.

3. The guide wire assembly as set forth in claim 2, wherein, when said base-end angular section passes through said pipe body as said guide wire is delivered from an outlet of said storage section, a state in which a direction of said-base end angular section is aligned with a direction for maximizing a width of the cross section of said hollow section, as viewed in a longitudinal direction of said pipe body, is maintained dynamically stable by elasticity of said base-end angular section, whereby a direction of said front-end angular section is maintained at a predetermined orientation with respect to said storage section.

4. The guide wire assembly as set forth in claim 2, wherein said hollow section of said pipe body has an elliptic cross sectional shape.

5. The guide wire assembly as set forth in claim 2, wherein said pipe body has a groove formed on an inside surface along the longitudinal direction thereof, and wherein the width of said hollow section is maximized in a direction of said groove.

6. The guide wire assembly as set forth in claim 2, wherein the cross sectional shape of said hollow section of said pipe body is such that a portion thereof in a circumferential direction of a circle is enlarged toward the outside in a radial direction.

7. The guide wire assembly as set forth in claim 2, wherein the cross sectional shape of said hollow section of said pipe body is such that a portion thereof near a central portion of one or both of shorter sides of a rectangle is enlarged toward the outside.

8. The guide wire assembly as set forth in claim 1, wherein said pipe body is disposed in a substantially annularly wound state.

9. The guide wire assembly as set forth in claim 1, wherein a distance from said main body section or an extension line thereof to a farthest part of said base-end angular section in a natural state is greater than a maximum inside diameter of said pipe body.

10. The guide wire assembly as set forth in claim 2, wherein said pipe body is disposed in a substantially annularly would state.

11. The guide wire assembly as set forth in claim 2, wherein a distance from said main body section or an extension line thereof to a farthest part of said base-end angular section in a natural state is greater than a maximum inside diameter of said pipe body.
